(19) United States
(12) Patent Application Publication
(10) Pub. No.: US 2010/0043523 A1
(43) Pub. Date: Feb. 25, 2010

(54) METHOD AND DEVICE FOR TESTING THE MEASURING ACCURACY OF A MEASURING DEVICE

(75) Inventor: Tryggve Hemmingsson, Sollentuna (SE)

Correspondence Address: MORRISON & FOERSTER LLP, 425 MARKET STREET, SAN FRANCISCO, CA, 94105-2482, US

(73) Assignee: AEROCRINE AB, Solna (SE)

(21) Appl. No.: 12/525,306

(22) PCT Filed: Jan. 28, 2008
(86) PCT No.: PCT/SE2008/050105

§ 371 Date: Oct. 29, 2009

(30) Foreign Application Priority Data
Feb. 1, 2007 (SE) 0700260-3

(51) Int. Cl. G01N 33/497

(52) U.S. Cl. 73/23.3

(57) ABSTRACT

A method for testing the measuring accuracy of a measuring device (7) for gas measurements, comprising a gas inlet opening (70), comprising the steps; connecting a special reference gas, having a known concentration and having a controlled flow rate and pressure, where the flow rate and the pressure are controlled to be within certain limits, to the gas inlet opening (70); and evaluating the reading in relation to the known concentration of the special reference gas. A control device (1), for use in the method, for controlling the pressure and flow rate of a gas source connected the control device (1) so as to assure a controlled pressure and flow rate at a gas output (17) of the control device (1), which comprises a flow rate controlling element (11) and a pressure controlling element (13) connected between the gas input (10) and the gas output (17).

### TECHNICAL FIELD

The present invention relates to the field of gas measurements, and more specifically to the field of testing the measuring accuracy of a measuring device for gas measurements.

The present invention also relates to a control device.

### BACKGROUND OF THE INVENTION

For testing the measuring accuracy of a measuring device for gas measurements it is known to use a special reference gas with a known concentration of the gas to be measured by the measuring device. For example, if the measuring device is used to measure the concentration of nitrogen monoxide in exhaled breath, a special reference gas with a known concentration of nitrogen monoxide in nitrogen is used. That is, nitrogen is the bulk gas with a known concentration of nitrogen monoxide. This special reference gas is stored in compressed form in a gas cylinder and is fed to the measuring device, often via a gas fitting and a pressure regulator. The reading of the measuring device is then compared with the known concentration of the gas to be measured in the special reference gas.

One type of pressure regulator that is used in the known art comprises two pressure meters, a valve for manually adjusting the output pressure of the pressure regulator and a stop valve at the output of the pressure regulator. Hence, the pressure regulator only allows an output pressure to be manually set, without further regulating possibilities. Consequently, the compressed gas is fed to the measuring device and to the gas sensor in a relatively uncontrolled way.

In several of the known measuring devices for gas measurements compressed gas can be fed with a relatively high pressure and in an uncontrolled way to the measuring device. Hence, the measuring accuracy of the measuring device can be tested by feeding gas from a gas cylinder containing a special reference gas in compressed form via a relatively simple pressure regulator to the gas sensor.

However, there are also measuring devices requiring that the gas is fed to the measuring device in a controlled way when the measuring device is used for gas measurements. Consequently, when testing the measuring accuracy of such a measuring device it is not possible to feed gas in a uncontrolled way to the measuring device. As an example some measuring devices using an electrochemical cell as gas sensor can be mentioned. The uncontrolled flow of gas from the pressure regulator would create conditions leading to the measuring device giving an inaccurate measurement result.

### SUMMARY OF THE INVENTION

Hence, there is a need for a technique enabling the use of special reference gas in compressed form also for measuring devices of the above mentioned type.

One aim of the present invention is to provide a technique as mentioned above.

This aim is achieved by the method according to independent claim 1 and further by the device according to independent claim 5.

According to the solution of the invention as defined in independent claim 1 there is provided a method for testing or checking the measuring accuracy of a measuring device for gas measurements where the measuring device includes a gas inlet opening.

The method comprises the step of connecting a special reference gas to the gas inlet opening of the measuring device. The special reference gas has a known concentration of the gas to be measured and has a controlled flow rate and pressure, where the flow rate and the pressure are controlled to be within certain limits. The flow rate may be in the range of 10 ml/s-10 l/s.

The method further comprises the evaluation of the reading, or the displayed value, of the measuring device in relation to the known concentration of the special reference gas.

An advantage of this method is that the special reference gas can be fed to the measuring device with a controlled flow rate and pressure. This enables the measuring device to measure the concentration of the gas to be measured in the special reference gas with a high accuracy.

This is important since the special reference gas is used to test or check the measuring accuracy of the measuring device. If the measuring device would be fed with the special reference gas in such a way as to create unsuitable measuring conditions for the measuring device this could lead to the situation that the reading or displayed value of the measuring device shows a deviation from the nominal concentration of the special reference gas that is interpreted as an error in the measuring device. But in reality the error to a large extent is caused by the fact that the special reference gas is fed to the measuring device in an inappropriate way.

According to the solution of the invention as defined in independent claim 5 there is provided a control device for use in the method according to the invention. The control device is used for testing the measuring accuracy of a measuring device for gas measurements and controls the pressure and flow rate of a gas source connected to an input connection of the control device so as to assure a controlled pressure and flow rate at a gas output connection of the control device. The control device comprises a flow rate controlling element and a pressure controlling element, both connected between the gas input connection and the gas output connection.

An advantage of this control device is that a measuring device for gas measurements can be fed with a controlled flow rate and pressure of a special reference gas. This enables the measuring device to measure the concentration of the gas to be measured in the special reference gas with a high accuracy.

Further advantageous embodiments of the invention are defined in the dependent claims.

### BRIEF DESCRIPTION OF THE DRAWINGS

In the following the method and the device according to the invention will be described by way of example with reference to the accompanying drawings wherein;

FIG. 1 shows the principal design of the control device 1.

FIGS. 2A and 2B show the flow rate controlling element 11 in a normal and in an exploded view.

FIG. 3 shows one example of a measuring device 7 with which the device and the method according to the invention can be used.

FIG. 4 shows an example of an input filter that can be used with the method and device according to the invention.

### DETAILED DESCRIPTION OF THE INVENTION

The method and the device according to the invention are applicable to any measuring device for gas measurements. However, in some of the examples described in the following, the method and the device according to the invention will be described in relation to a particular measuring device to facilitate the understanding of the invention. The measuring device in question is one model of the NIOX MINO®, marketed by Aerocrine AB, Solna, Sweden, which is a measuring device for diagnostic gas measurements, measuring for example the concentration of nitrogen monoxide in exhaled breath.

A control device 1 is intended to be coupled to some sort of source of special reference gas in compressed form. One possible such source is a gas cylinder with compressed special reference gas. For a measuring device 7 measuring the concentration of nitrogen monoxide a special reference gas with a concentration of 200 ppb nitrogen monoxide in nitrogen can be used. The pressure for a full gas cylinder can typically be 170 bar and the gas cylinder is preferably coupled to the control device 1 via a pressure regulator, often present at the gas cylinder, which enables the manual limitation of the pressure at the output of the pressure regulator.

In one embodiment of the invention the control device 1 comprises the following parts:

a housing 20, a gas input connection 10, a gas output connection 17, a flow rate controlling element 11 and a pressure controlling element 13.

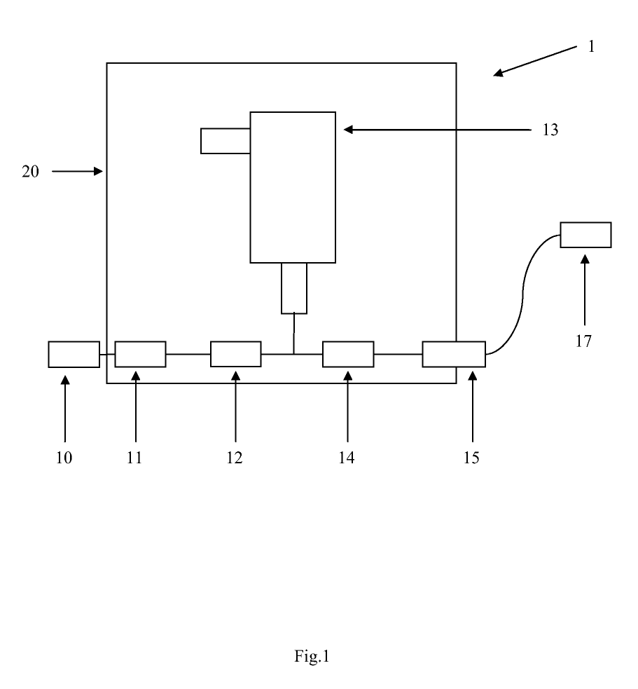

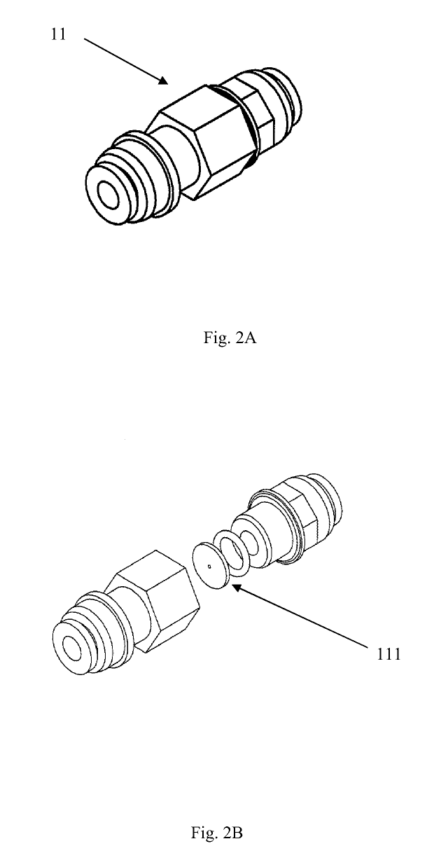

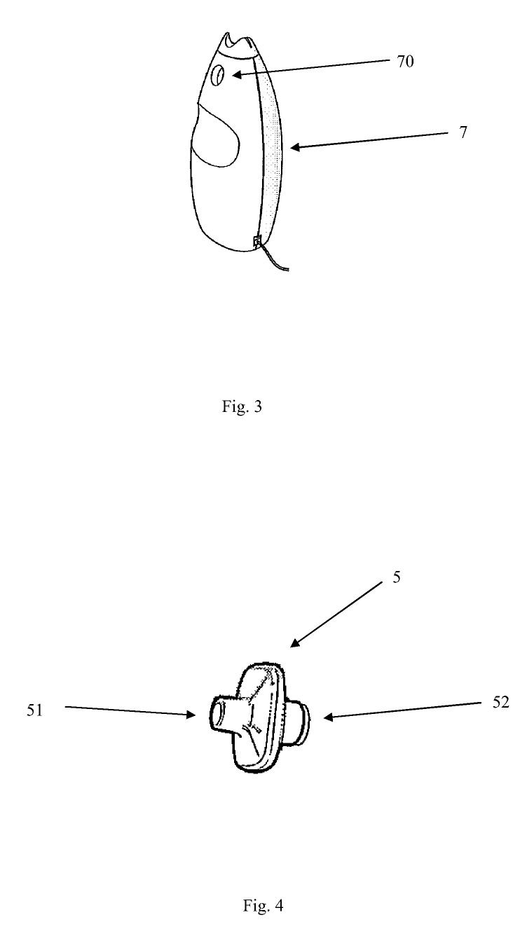

As can be seen in FIG. 1 the gas input connection 10 of the control device 1 is connected to the flow rate controlling element 11 which in turn is connected to a first connection 12 of the pressure controlling element 13 via a piece of tubing or the like. The pressure controlling element 13 is connected with its second connection 14 to an intermediate coupling 15 via a piece of tubing or the like. The intermediate coupling 15 is connected to the gas output connection 17 via a flexible hose, flexible tube, or the like.

The flow rate controlling element 11 comprises a coupling including a restriction washer 111. The pressure controlling element 13 is of a over flow-type. That is, when the pressure in the pipe to which the pressure controlling element 13 is connected, exceeds a certain limit or threshold, the pressure controlling element 13 opens an internal valve and releases gas until the pressure in the pipe decreases to a level below the threshold. For a given input pressure at the gas input connection 10 of the control device 1 the through hole of the restriction washer 111 is adapted to the amount of gas that the pressure controlling element 13 releases when it controls the pressure. This adaptation is done so that a specific flow rate, or a specific range for the flow rate, can be met at the gas output connection 17 of the control device 1 at the same time as the pressure is kept at a certain value, or within certain limits, by the pressure controlling element 13.

The flow rate controlling element 11 and the pressure controlling element 13 are designed so as to give a flow rate and pressure at the gas output connection 17 of the control device 1 that are within certain limits. The intervals for the output flow rate and the output pressure of the control device 1 are adapted to the specific measuring device 7 with which the control device 1 is to be used.

To illustrate the design principle of the control device 1 a preferred embodiment relating to a particular measuring device 7 (one model of the NIOX MINO®) will now be given. The pressure regulator on the gas cylinder containing a special reference gas is adjusted to an output pressure of around 2 bar, which is a suitable pressure for the design of the flow rate controlling element 11 and the pressure controlling element 13 to give reasonable dimensions and performance requirements for these parts. However, other output pressures are of course possible, for example in the range of 1-10 bar, more preferably 1-3 bar.

With an input pressure of approximately 2 bar at the gas input connection 10 of the control device 1, the flow rate controlling element 11 is dimensioned so as to give a flow rate after the flow rate controlling element 11 in the range of 50-1000 ml/s, more preferably 200-800 ml/s in combination with a typical pressure controlling element 13.

In one particular example the through hole in the restriction washer 111 of the flow rate controlling element 11 is calculated to 0.8 mm in diameter which gives a flow rate of approximately 200-300 ml/s after the flow rate controlling element 11 when a pressure of approximately 2 bar is applied at the gas input connection 10 of the control device 1. The pressure controlling element 13 is dimensioned to give a pressure at the gas output connection 17 of the control device 1 that is approximately 15 hPa when the flow rate after the flow rate controlling element 11 is in the range of approximately 200-300 ml/s. With the conditions mentioned in the foregoing a flow rate in the range of 10 ml/s-500 ml/s and a pressure in the range of 5-30 hPa at the gas output connection 17 of the control device 1 can be assured. Preferably the flow rate is around 50 ml/s and the pressure around 15 hPa at the gas output connection 17 of the control device 1. A flow rate of approximately 50 ml/s and a pressure of approximately 15 hPa has been found to be suitable for some measuring devices.

With other diameters of the through hole in the restriction washer 111 and other dimensioning of the pressure controlling element 13 other values of the flow rate and pressure at the gas output connection 17 of the control device 1 can be achieved. For example can pressures in the interval of 5-50 hPa and flow rates in the interval of 10 ml/s-10 l/s be suitable for many applications. The pressure controlling element 13 may be a Positive End Expiratory Pressure Valve (PEEP-Valve).

Of course the mentioned embodiment is just one example of how the control device 1 can be dimensioned.

The inventive idea lies in the careful and precise dimensioning of the flow rate controlling element 11, the pressure controlling element 13 and the connecting tubes and other components in the control device 1 in relation to each other and to a certain input gas pressure at the gas input connection 10 of the control device 1. For example it is important to adapt the inner diameter and the length of the tubing in the control device 1 to the characteristics of the pressure controlling element 13. The pressure controlling element 13 has a certain hysteresis and this is one reason why the inner diameter and the length of the tubing in the control device 1 have to be adapted to the pressure controlling element 13. This careful and precise dimensioning is necessary to create a controlled gas pressure and a controlled flow rate at the gas output connection 17 of the control device 1. Both the flow rate and the pressure are within a specified range so as to enable the measuring device 7 to which the control device 1 is connected to give an accurate measurement of the concentration, or other parameters, of the gas to be measured in the gas mixture fed to the measuring device 7.

In the method according to the invention a special reference gas with a known concentration of the gas to be measured is connected to a measuring device 7 for gas measurements. The special reference gas is controlled concerning the flow rate and the pressure. The flow rate and the pressure of the special reference gas are preferably controlled to be within certain limits or intervals.

In one embodiment, the following steps are performed in the method according to the invention:
1. The output connection of the pressure regulator of a gas cylinder with special reference gas is connected to the gas input connection 10 of the control device 1 via a hose or tube.
2. The output pressure of the pressure regulator is adjusted to a suitable value for the measuring device 7 in question and the output valve of the pressure regulator is opened.
3. The output connection 17 of the control device 1 is connected to the gas inlet opening 70 on a measuring device 7 for gas measurements.
4. The reading or the displayed value of the measuring device 7 is noted or recorded and compared with, and/or evaluated in relation to, the known concentration of the special reference gas. The concentration of the special reference gas is usually indicated on the gas cylinder certificate.

It is conceivable that for some types of measuring devices 7 it is necessary to bring the measuring device 7 to a state of being ready to be connected to a gas source, i.e. the control device 1, before the output connection 17 of the control device 1 can be connected to the gas inlet opening 70 on the measuring device 7 (i.e. before step 3.).

The conditions to be met for the test to be satisfactory differ between different measuring devices. Often a test is defined as satisfactory when the reading or the displayed value of the measuring device lies within a specified interval around the indicated concentration for the special reference gas. Such an interval can for example be specified as the indicated concentration of the special reference gas +/−5%, +/−10% or +/−15%, just to mention some examples.

For one type of measuring device 7 the following steps are performed in the method according to the invention:
1. The pressure regulator of a gas cylinder with special reference gas is flushed 10-15 times. To flush such a regulator is a known procedure and does not need to be described in detail.
2. The output connection of the pressure regulator of the gas cylinder with special reference gas is connected to the gas input connection 10 of the control device 1 via a hose or tube.
3. The output pressure of the pressure regulator is adjusted to a pressure between approximately 1 and 3 bar, preferably approximately 2 bar after which the output valve of the pressure regulator is closed.
4. Check that the measuring device 7 is ready for use.
5. Optionally an input filter 5 for filtering out moist, virus, microbes, bacteria and the like, having an input connection end 51 and an output connection end 52, can be connected with its output connection end 52 to the gas inlet opening 70 of the measuring device 7.
6. The output valve of the pressure regulator is opened.
7. A human empties hers/his lungs, and inhales deeply to total lung capacity through the gas inlet opening 70 of the measuring device 7.
8. The output connection 17 of the control device 1 is connected to the gas inlet opening 70 on the measuring device 7, or if an input filter 5 is used, the output connection 17 of the control device 1 is connected to the input connection end 51 of a new input filter 5, in order to assure no transmittal of unwanted material, such as bacteria and viruses.
9. Check that the measuring device 7 indicates that the pressure of the connected gas source (the output connection 17 of the control device 1) is ok. If the measuring device 7 indicates that the pressure is too low the output pressure of the pressure regulator of the gas cylinder is raised a bit, e.g. with 0.5 bar and it is checked if the measuring device 7 indicates that the pressure is ok. If the measuring device 7 indicates that the pressure is too high the output pressure of the pressure regulator of the gas cylinder is lowered a bit, e.g. with 0.5 bar, and it is checked if the measuring device 7 indicates that the pressure is ok.
10. When the measuring device 7 indicates that the pressure and timing, or similar characteristics, for the gas supply is ok the output connection 17 of the control device 1 is disconnected from the measuring device 7, or alternatively, from the input connection end 51 of the input filter 5. With timing is meant that the measuring device 7 can require that a gas source should be connected to the measuring device 7 within a certain period of time. This time period can be defined by the software for example of the measuring device 7.
11. The reading or the displayed value of the measuring device 7 is noted or recorded and compared with, and/or evaluated in relation to, the known concentration of the special reference gas.

The test is defined as satisfactory when the reading or the displayed value of the measuring device 7 lies within a specified interval around the indicated concentration for the special reference gas. Such an interval can for example be specified as the indicated concentration of the special reference gas +/−5%, +/−10% or +/−15%, just to mention some examples.

Although the method and the device according to the invention have been exemplified with some examples relating to a measuring device for the measurement of the concentration of nitrogen monoxide in exhaled breath, of course other applications are possible. The method and the device according to the invention are for example equally well suited when it comes to applications relating to the measurement of other gases in exhaled breath, in air or in other gas mixtures. Examples of such other gases are oxygen, ethanol, acetone, carbon dioxide, carbon monoxide, hydrogen or nitrogen dioxide. The method and the device according to the invention are also applicable to measuring devices measuring other parameters of a gas than its concentration.

1. A method for testing the measuring accuracy of a measuring device for gas measurements, the measuring device comprising a gas inlet opening, the method characterised in;
a. connecting a special reference gas, having a known concentration of the gas to be measured and having a controlled flow rate and pressure, where the flow rate and the pressure are controlled to be within certain limits, the flow rate being in the range of 10 ml/s-10 l/s, to the gas inlet opening; and
b. evaluating the reading of the measuring device in relation to the known concentration of the special reference gas.

2. The method according to claim 1, further comprising the special reference gas having a pressure, which preferably is in the range of 5-30 hPa.

3. The method according to claim 1, further comprising;
a. a human inhaling through the gas inlet opening, the gas inlet opening thereby also functioning as a gas outlet opening, prior to connecting the special reference gas to the gas inlet opening.

4. The method according to claim 3 further comprising;
a. connecting an input filter, for filtering out moist, virus, microbes, bacteria and the like, having an input and an output connection end, with its output connection end to the first gas inlet opening; and
b. the human inhaling through the input connection end of the input filter, prior to connecting the special reference gas to the input connection end of a new input filter that has been connected to the measuring device.

5. A control device, for use in the method according to claim 1, for controlling the pressure and flow rate of a gas source connected to an input connection of the control device so as to assure a controlled pressure and flow rate at a gas output connection of the control device, the control device comprising; a flow rate controlling element and a pressure controlling element connected between the gas input connection and the gas output connection.

6. The control device according to claim 5 wherein the flow rate controlling element is connected between the gas input connection and the pressure controlling element.

7. The control device according to claim 5 wherein the pressure controlling element comprises a positive end expiratory pressure valve.

8. The control device according to claim 5 wherein the flow rate controlling element comprises a restriction washer.
